(19) United States
(12) Patent Application Publication
(10) Pub. No.: US 2010/0260737 A1
(43) Pub. Date: Oct. 14, 2010

(54) COMBINATION BETWEEN AN ISOTHIOCYANATE AND LEVODOPA FOR PARKINSON'S DISEASE TREATMENT

(76) Inventors: Andrea Tarozzi, Bologna (IT); Fabiana Morroni, Bologna (IT); Giorgio Cantelli Forti, Bologna (IT); Patrizia Hrelia, Bologna (IT)

Correspondence Address: NIXON & VANDERHYE, PC, 901 NORTH GLEBE ROAD, 11TH FLOOR, ARLINGTON, VA, 22203, US

(21) Appl. No.: 12/808,830

(22) PCT Filed: Dec. 17, 2008
(86) PCT No.: PCT/IB08/55377

§ 371 Date: Jun. 17, 2010

(30) Foreign Application Priority Data
Dec. 20, 2007 (IT) RM2007A000668

(51) Int. Cl. A61K 38/44; A61K 36/31; A61K 31/26; A61K 31/7028; A61P 25/16

(52) U.S. Cl. 424/94.4; 424/755; 514/515; 514/24

(57) ABSTRACT

A combination between levodopa and an extract derived from a vegetable of the Cruciferae family or Brassica genus, this extract containing sulforaphane (4-(methylsulfinyl)butyl isothiocyanate), is disclosed. This combination is useful for the treatment of Parkinson's disease and in particular, for on-off and wearing-off episodes.

This invention relates to the pharmaceutical and nutritional fields, and in particular it relates to a combination between levodopa and natural compounds, isothiocyanates, which exert a synergistic neuroprotective effect with levodopa.

### BACKGROUND OF THE INVENTION

Levodopa (3,4-dihydroxyphenylalanine) or L-DOPA, an immediate precursor of dopamine, is the most effective medicine for relieving the symptoms of Parkinson's disease (PD). The occurrence of motor complications is the major problem in the long-term management of patients with PD, in particular the wearing off and on-off phenomena which can induce severe impairments and reduce therapy effectiveness. About 90% of patients show motor impairments after 10 or more years of L-DOPA treatment. These adverse reactions are most strongly related to disease duration, dose and duration of levodopa treatment (Schrag A. and Quinn A., Brain, 2000, 123, 2297-2305).

Several pathogenetic events may contribute to motor impairments caused by L-DOPA, such as the progressive degeneration of dopaminergic neurons and the reduced possibility of L-DOPA storage. In particular, intermittent dopaminergic stimulation, due to L-DOPA administration, may be associated with motor complications (Chase T N and Oh J D., Ann. Nerol. 2000, 47:S122-S129). Recent studies show that oxidant formation, following L-DOPA metabolism, could cause dopaminergic neuronal death (Smith T S. et al., Neuroreport 1994, 5, 1009-1011; Pardo B. et al., Brain Res. 1995, 682, 133-143; Nakao N., Brain Res. 1997, 777, 202-209). The limits of L-DOPA treatment are therefore both interactions between the drug and the neuronal circuit and intrinsic drug toxicity (Obeso J A. Et al., Trends Neurosci. 2000, 23, S8-S19).

The current clinical strategies to prevent or to delay motor impairments include delaying the start of L-DOPA therapy, the use of low dose therapy, the administration of drugs, which exert continuous dopaminergic stimulation and the decrease of dopaminergic cell death. The recent national and international guidelines for PD treatment suggest the use of L-DOPA when the disease symptoms cause functional impairments.

Epidemiological evidences suggests that dietary antioxidants, like vitamins and polyphenols, may act as disease-modifying neuroprotective compounds, by reduction of neuronal death in both in vitro and in vivo models (Ramassamy C. Eur. J. Pharmacol. 2006, 545, 51-64). Other dietary compounds, besides the well known antioxidants, may represent treatment avenues for chronic neurodegeneration.

Sulforaphane (4-(methylsulfinyl)butyl isothiocyanate or SUL) is a glucosinolate-derived isothiocyanate found in cruciferous vegetables. Isothiocyanates are obtained from vegetables such as broccoli, cauliflower and Brussel sprouts and their detoxicant and anticancer activity has been described (Hoist B. and Williamson G., Nat. Prod. Rep., 2004, 425-447). Among the isothiocyanates, SUL has a specific biological profile at neuronal level to become a promising candidate for the therapy of neurodegenerative diseases (Konwinski R R. et al., Toxicol. lett., 2004, 343-355). SUL was submitted to a preliminary phase I study which showed the absence of toxicity in humans (Shapiro T A. Et al., Nutr. Cancer. 2006, 55, 53-62).

Recent studies have demonstrated potential neuroprotective effects of SUL in various neurodegenerative models. In particular, SUL and its glucosinolate consumption reduce inflammation and ischemia in the CNS, this result proves that SUL can cross the blood brain barrier and it can counteract post-traumatic cerebral edema (Noyan-Ashraf M. et al., Nutr. Neurosci., 2005, 101-110; Zhao J. Et al., J. Neurosci. Res. 2005, 82, 499-506, Neurosci. Lett. 2006, 393, 108-112; US2006/0116423A1). As with other isothiocyanates, SUL's neuroprotective mechanism of action is not yet known.

Recent in vitro findings have shown that prolonged SUL treatment protects neurons against H₂O₂ damage and against 6-hydroxydopamine but it does not show any effect against another neurotoxin used as a PD model, 1 methyl-4-phenyl-1,2,3,6-tetrahydropyridine. SUL may exert its action by modulating the gene expression of phase II enzymes, which are known for their antioxidant and detoxicant action (Kraft et al., J. Neurosci. 4:1101-1112, 2004; Han et al., J. Pharmacol. Exp. Ther. 321:249-256, 2007). In particular, these results highlight that SUL prevents the initial phase of the neurodegenerative process, and neuroprotective effects of SUL could be ascribed to the increase of cellular antioxidant defenses. The ability of SUL to directly counteract and to rescue neuronal damage has not yet been confirmed.

Although antioxidants and supplements could theoretically help in the treatment of PD, clinical studies have demonstrated that tocopherol, coenzyme Q10, and glutathione appear to have a limited role in the prevention or treatment of PD (Weber C A. Ann. Pharmacother. 2006, 40, 935-938). One of the reasons for this failure is probably the short “therapeutic window” of direct antioxidants in patients with neurodegenerative diseases. In fact, oxidative damage is usually considerable and the degenerative process has already started at the time of the diagnosis.

Consequently, antioxidants have a marginal role in the field of neuroprotection and in particular in PD therapy.

Therefore, the problems of the neurodegenerative process and their complications induced by long-term L-DOPA therapy have not yet been solved.

This invention aims to solve these and related problems.

### SUMMARY OF THE INVENTION

It has now surprisingly been found that the combination between SUL and L-DOPA exerts an effective neuroprotective activity. This effect is not only in contrast with the results of antioxidants as neuroprotective agents but we also indicate a synergistic effect.

In particular, the combination of L-DOPA and SUL shows neuroprotective effects against oxidative stress.

Therefore, an object of this invention is the combination between SUL and L-DOPA.

This invention can ameliorate the ratio risk/benefit associated with L-DOPA therapy, and can prevent and delay the neurodegeneration induced by L-DOPA.

In particular, the combination with SUL protects neurons against L-DOPA-induced oxidative damage and blocks the progression of the process.

SUL counteracts L-DOPA toxicity and we don't therefore need to modify the chemical structure of L-DOPA and all the preclinical and clinical trials necessary for the approval of a new molecule can be avoided. Pinnen et al. demonstrate that molecules derived from L-DOPA and antioxidant molecules, such as glutathione and lipoic acid, decrease the oxidative stress caused by L-DOPA autoxidation and metabolism at plasma level. They also increase dopamine concentration in the CNS by acting as prodrugs (Di Stefano A. et al., J. Med. Chem., 2006, 49, 1486-1493; Pinnen F. et al., J. Med. Chem., 2007, 50, 2506-2515). It has not, however, been shown whether these polyfunctional compounds decrease the pro-oxidant effects of L-DOPA or dopamine which are more concentrated in the CNS.

The combination of this invention is used to prepare drugs or nutritional products (nutraceuticals) valuable in PD treatment. This application and the composition of this product is another object of the invention.

A further object of this invention is also the combination, described above, with an inhibitor of monoamine-oxidase B, MAOB (Selegiline) or cathecol-O-methyltransferase, COMT (Entecapone and Tolcapone).

These and other objects of the present invention will be described in more detail here, also using examples and figures.

### DESCRIPTION OF THE INVENTION

As mentioned above, this invention is founded on the discovery of the synergistic effect of SUL and L-DOPA combination.

The present invention also has other potential applications.

It is not necessary to isolate SUL, in fact it is possible to obtain the same results using glucosinolate. The isolation of SUL and the related glucosinolate is already known (Vaughn S F. E Berhow M A., Industrial Crops and Products, 2005, 21:193-202; Rochfort S. et al., J. Chromatogr. A. 2006, 1120:205-210; Liang H. et al., J. Agric. Food Chem. 2007, 55:8047-8053), so those details are not reported here for the realization of the present invention.

The same results are obtained using L-DOPA in combination with vegetable extracts which include SUL or its glucosinolate.

Examples of vegetable extracts for the present invention are the ones obtained from plants of the Cruciferae family and Brassica genus, such as broccoli, cabbage, cauliflower, Brussel sprouts, turnip, celery, mustard, radish. These extracts and the process to obtain them are also known (PNAS 1997, 94, 10367-10372).

Therefore, the combination of the present invention can also be realized with sulforaphane glucosinolate or vegetable extracts containing it.

Taking the above into account, other isothiocyanates with neuroprotective activity have same results. Examples of isothiocyanates are:

| Glucosinolate (precursor) | Isothiocyanate |
|---|---|
| Glucocapparin | Methylisothiocyanate |
| Glucoibervirin | 3-(methylthio)propyl isothiocyanate |
| Glucoerucin | 4-(methylthio)butyl isothiocyanate |
| Glucoiberin | 3-(methylsulfinyl)propyl |
| | isothiocyanate |
| Glucocheirolin | 3-(methylsulfonyl)propyl |
| | isothiocyanate |
| Glucoerysolin | 4-(methylsulfonyl)butyl |
| | isothiocyanate |
| Sinigrin | Allyl(2-propenyl) isothiocyanate |
| Gluconapin | 3-butenyl isothiocyanate |
| Progoitrin | 2-hydroxy-3-butenylisothiocyanate |
| Glucobrassicanapin | 4-pentenyl isothiocyanate |
| Glucoraphenin | 4-(methylsulfinyl)-3-butenyl |
| | isothiocyanate |
| Glucotropaeolin | Benzyl isothiocyanate |
| | 2-hydroxybenzyl isothiocyanate |
| Gluconasturtin | 2-phenylethyl isothiocyanate |
| Glucobrassicin | 3-indolylmethyl isothiocyanate |
| 4-methoxyglucobrassicin | 4-methoxy-3-indolylmethyl |
| | isothiocyanate |
| Neoglucobrassicin | 1-methoxy-3-indolylmethyl |
| | isothiocyanate |

The present invention is based on the use of a combination between neuroprotective isothiocyanates, also as glucosinolate or vegetables extracts in which they can be found, in particular those derived from the Cruciferae family and Brassica genus, in a preparation for human administration.

This composition, which is a further object of the present invention, is prepared following the general knowledge in the field and doesn't require any particular instruction from the present inventors, merely the knowledge for the preparation of the single components.

General knowledge about the formulation of preparation for human administration is available in manuals, such as the latest edition of Remington's Pharmaceutical Sciences, or similar manuals and in the European and Italian Pharmacopoeia.

This composition of the present invention can take the form of a drug or dietary supplement, according to the concentration of its components and to marketing drug regulatory rules of each country in which it will be sold. This distinction is anyway not important for the present invention, because the frequency of administration, the concentration and route of administration are decided by each doctor, who can choose in accordance with the patient's conditions and the severity of the disease.

The aim of the present invention is to disclose a composition which allows the treatment of PD by using L-DOPA, which is however the drug of choice, but without the occurrence of wearing off and on-off episodes, thanks to the neuroprotective activity of the isothiocyanate. The effects of the invention are based on the synergism between L-DOPA and isothiocyanate, which was unexpected from prior art.

The composition of the present invention can be administered in all the known forms, enteral or parenteral, solid, semi-solid or liquid. Examples of formulation are tablets, capsules, also controlled-release form, suspensions, emulsions and solutions, such as syrup and elixir. Injectable forms, like solutions, suspensions and emulsions, also in depot form, controlled-release transdermic systems are also included.

Vegetable extracts are obtained by traditional methods and they could be liquid or dried. The definition of extract is in the European and Italian Pharmacopoeia.

The administration of the two components can occur at the same or at different times, as shown in the following results. The sequence of administration will be decided by each doctor. For example, the combination of the present invention can be in the same preparation, such as in a tablet or capsule, or in separate forms, which can be administrated simultaneously or in sequence, according to the medical prescription. The single preparation can be a tablet, such as a tablet which releases the component at different times.

Neuroprotective agents can be administrated before, during or after L-DOPA treatment, so there are three therapeutic windows in which the agent can counteract the damage induced by L-DOPA.

The doses of the single components of the combination will be obtained by clinical studies. Each component is already known for toxicity and efficacy, so the drug development expert will not have any difficult in studying the synergistic effects of the composition of the present invention.

In vitro experiments show neuroprotective and synergistic effects using SUL (0.63 μM) 40 times less concentrated than L-DOPA (25 μM). The concentrations used for the experiments are equivalent to the plasma levels obtained in humans, after administration of broccoli extract or L-DOPA (Ye L. et al., Clin. Chim. Acta 2002, 316:43-53; Dethy S., Clin. Chem. 1997, 43:740-744).

The invention is now illustrated by the following example and figures in which:

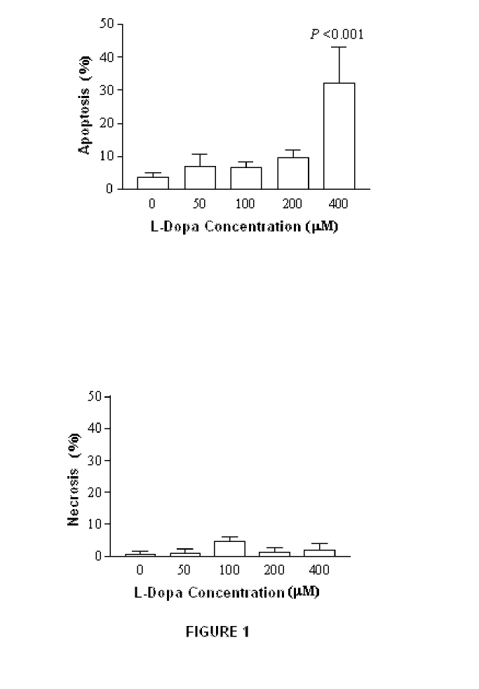

FIG. 1 shows apoptosis and necrosis in SH-SY5Y cells after treatment with various concentrations of L-DOPA. Results are reported as average ±standard deviation of three different experiments.

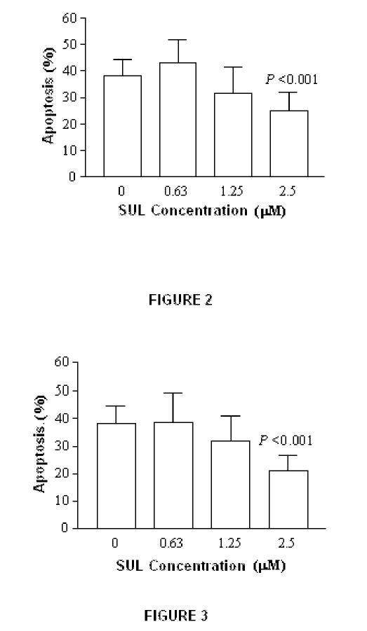

FIG. 2 shows apoptosis in SH-SY5Y cells after co-treatment with L-DOPA (400 μM) and various concentrations of SUL. Results are reported as average ±standard deviation of three different experiments.

FIG. 3 shows apoptosis in SH-SY5Y cells after L-DOPA (400 μM) treatment and post-treatment with various concentrations of SUL. Results are reported as average ±standard deviation of three different experiments.

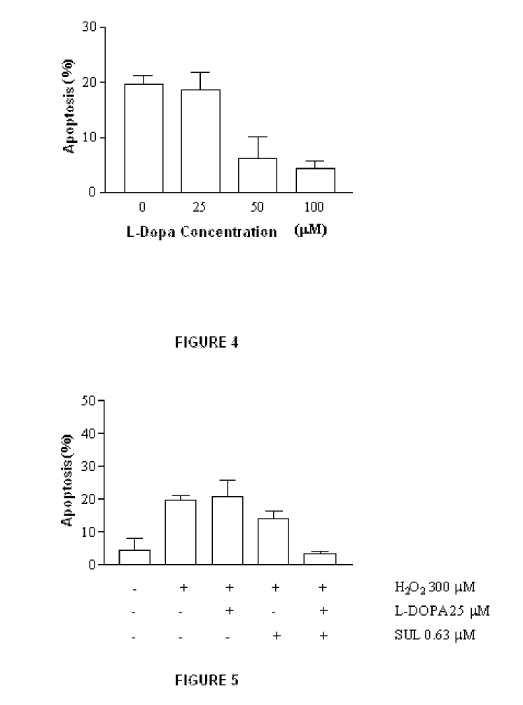

FIG. 4 shows apoptosis in SH-SY5Y cells pre-treated with various concentrations of L-DOPA and after treated with H₂O₂ (300 μM). Results are reported as average ±standard deviation of one representative experiment.

FIG. 5 shows apoptosis in SH-SY5Y cells pre-treated with L-DOPA (25 μM) and SUL (0.63 μM) and after treated with H₂O₂ (300 μM). Results are reported as average ±standard deviation of one representative experiment.

### EXAMPLE

### Pharmacological Assays

In order to evaluate the neuroprotective effects of SUL against L-DOPA-induced neurotoxicity, an experimental approach using SH-SY5Y cells, a dopaminergic neuronal cell lines, was applied. A pulse/chase treatment has been used, which means a short exposure of neurons to L-DOPA and then it is removed to allow the activation of neuronal cell death mechanisms. In particular, apoptotic events and necrosis are detected with Annexin-V/propidium iodide (PI) double-staining system after 15 h of 3 h treatment with L-DOPA. (Lai C T. et Yu P H., Biochem. Pharmacol. 1997, 53:363-372).

The neuroprotective activity of new molecules can be determined at three different times with the pulse/chase treatment: before, during and after the exposure to L-DOPA. These therapeutic windows allow defining the period within which the administration of a molecule can exert its neuroprotective effects.

As reported in FIG. 1, treatment of SH-SY5Y cells with L-DOPA (50-400 μM) showed a significant increase of apoptotic cell death with 400 μM of L-DOPA. At the same time, necrotic death does not increase in the same conditions.

Co-treatment of neuronal cells with SUL (0.63-2.5 μM) and L-DOPA (400 μM) showed a dose-dependent inhibitory effect on L-DOPA-induced apoptosis (FIG. 2). To be sure that the neuroprotective effects are not caused by direct interaction with L-DOPA, the compound was added after the treatment with L-DOPA. The results also demonstrate that treatment of neuronal cells with 2.5 μM of SUL after L-DOPA treatment showed a significant decrease of apoptosis (FIG. 3).

It was also evaluated whether the combination could have synergistic effects against neuronal apoptosis induced by H₂O₂, an oxidant agent in the CNS. In particular, apoptosis is measured 15 h later than 3 h treatment with H₂O₂ (300 μM).

To determine the concentration of L-DOPA to associate with SUL, SH-SY5Y cells were pre-treated with low concentrations of L-DOPA (25-100 μM) for 24 h. As illustrated in FIG. 4, treatment of neurons with more than 50 μM of L-DOPA significantly decreased H₂O₂-induced apoptosis. This result could be ascribed to the neurohormesis phenomenon; some molecules at subtoxic doses activate adaptive cellular stress-response pathways in neurons.

The concentrations of SUL and L-DOPA, 0.63 and 25 μM respectively, used for the experiments, did not show any toxic effects in SH-SY5Y cells. FIG. 5 shows that pre-treatment of neurons with L-DOPA and SUL inhibits neuronal apoptosis induced by H₂O₂.

Taken together, these results demonstrate that SUL protects dopaminergic neurons against oxidative injury induced by high doses of L-DOPA and it also blocks the progression of the damage. Therefore SUL's neuroprotective effects could not be ascribed to the induction of the synthesis of antioxidant molecules and enzymes, but they could be due to the ability of SUL to interact with specific targets of L-DOPA damage.

Synergistic neuroprotective effects are also very interesting, especially for the low concentrations, highlighting an elevated specifity in the mechanisms of action.

1. A combination of levodopa and a vegetable extract obtained from a plant of Cruciferae family or Brassica genus.

2. A combination of levodopa and an isothiocyanate selected from the group consisting of: 4-(methylsulfinyl)butyl isothiocyanate, methyl isothiocyanate, 3-(methylthio)propyl isothiocyanate, 4-(methylthio)butyl isothiocyanate, 3-(methylsulfinyl)propyl isothiocyanate, 3-(methylsulfonyl)propyl isothiocyanate, 4-(methylsulfonyl)butyl isothiocyanate, allyl(2-propenyl) isothiocyanate, 3-butenyl isothiocyanate, 2-hydroxy-3-butenyl isothiocyanate, 4-pentenyl isothiocyanate, 4-(methylsulfinyl)-3-butenyl isothiocyanate, benzyl isothiocyanate, 2-hydroxybenzyl isothiocyanate, 2-phenyl ethyl isothiocyanate, 3-indolylmethyl isothiocyanate, 4-methoxy-3-indolylmethyl isothiocyanate and 1-methoxy-3-indolylmethyl isothiocyanate.

3. A combination according to claim 2, wherein the isothiocyanate is in glucosinolate form.

4. A combination according to claim 1, with the addition of a DOPAdecarboxylase inhibitor.

5. A combination according to claim 4, wherein DOPA-decarboxylase inhibitor is selected from the group consisting of Carbidopa and Benserazide.

6. A combination according to claim 5, further comprising a monoamine-oxidase B or catechol-O-methyl-transferase inhibitor.

7. A combination described in claim 6, wherein said inhibitor is Selegiline and the inhibitor of catechol-O-methyl-transferase is selected from the group consisting of Entecapone and Tolcapone.

8. A combination for human administration of claim 1.

9. Combination of claim 1, for use as medicament.

10. Combination of claim 1, for the treatment of Parkinson's disease.

11. Combination of claim 10 for the treatment of on-off and/or wearing off episodes.
